(19) United States
(12) Patent Application Publication
(10) Pub. No.: US 2019/0321154 A1
(43) Pub. Date: Oct. 24, 2019

(54) MULTIFUNCTIONAL HERNIA PATCH

(71) Applicant: TUBITAK, Ankara (TR)

(72) Inventors: Serdar SEZER, Kocaeli (TR); Ümran AYDEMIR SEZER, Kocaeli (TR); Hacer DOGAN, Kocaeli (TR); Ali AKTEKIN, Kocaeli (TR); Vildan SANKO, Kocaeli (TR); Selçuk HAYDANLI, Kocaeli (TR); Fugen AKER, Kocaeli (TR)

(73) Assignee: TUBITAK, Ankara (TR)

(21) Appl. No.: 16/310,454

(22) PCT Filed: Jun. 15, 2016
(86) PCT No.: PCT/IB2016/053534

§ 371 Date: Dec. 17, 2018

(51) Int. Cl. A61F 2/00; A61L 31/04; A61L 31/10; A61L 31/12; A61L 31/14

(52) U.S. Cl. CPC A61F 2/0063; A61L 31/048; A61L 2420/04; A61L 31/129; A61L 31/146; A61L 31/10

(57) ABSTRACT

A surgical implant with anti-adhesive, antibacterial and hemostatic properties to use in hernia repair. The implant includes:
a) Bilayer intraperitoneal mesh, having biocompatible, antibacterial, hemostatic and anti-adhesive properties including combination of biocompatible non-degradable or semi-degradable mesh. The mesh is prepared with blend system of biodegradable polyester-based polymers/chitosan mixture in hexafluoro isopropanol (HFIP) solvent and coated on polypropylene (PP) layer as nanofibers via electrospun technique; and,
b) Double and/or triple-layer extraperitonal composite mesh, was prepared with blend systems of biodegradable polyester-based polymers and/or polysaccharides formed on PP/polyester woven material via different coating methods.

### CROSS REFERENCE TO THE RELATED APPLICATIONS

This application is the national phase entry of International Application No. PCT/IB2016/053534, filed on Jun. 15, 2016, the entire content of which is incorporated herein by reference.

### TECHNICAL FIELD

This invention discloses composite meshes with anti-adhesive, antibacterial and hemostatic properties are used after intraperitoneal and extraperitoneal hernia surgery to accelerate tissue regeneration and prevent post-surgery complications. Electro-spinning and weaving methods are applied to design mesh composites.

### BACKGROUND

Membranes, acting as a physical barrier and preventing adhesion of tissues are called “Anti-adhesive membranes”. Adhesions of tissues are frequently observed after intraperitoneal and pelvic operations. Trauma, infection, pain and function disorder are other major problems for patients during healing process after such operations [Linsky et al., 1991, Alatsuzaki et al., 2014]. Especially after hernia surgery many of these complications are common and require additional medical treatment or even second surgery to remove mesh implant [Lim et al., 2008, Collage et al., 2010]. Therefore anti-adhesive membranes are employed during operations in order to prevent or diminish dangerous complications [Oesser et al., 2013, Obayan et al., 2013]. Biodegradable materials are generally preferred because they are removed from the body without medical intervention [Oesser et al., 2013]. Polysaccharides, proteins, oxidized regenerated cellulose, sodium carboxymethyl cellulose, dextran sulfate, hyaluronic acid, chondroitin sulfate, polyglycolic acid and polylactic acid can be used alone or in combinations [Lee et al., 2006]. However membranes from non-biodegradable materials are also common in clinical applications. These materials preventing adhesion may be designed in many different forms [Stopek et al., 2013], such as solution, gel or film [Kim et al., 2015]. Anti-adhesive membranes in solid form are preferable to gel, foam or liquid formulations because of practical fixing of material [Lee et al., 2006].

The basis of modern hernia surgery begins with the use of synthetic patch (polyamide patch) by Usher (Polyamide patch) in 1958. Afterwards, braided polyester patch, polypropylene mesh, expandable polytetrafluoroethylene (PTFE) patches were used and these products have been their placed in the historical process in the repair of abdominal wall hernias [Usher et al., 1963]. From the 1900's until today, varying patching materials have been introduced. In 1962, recurrence rate of 30-50% in the incisional hernia has started to decrease gradually with use of the monofilament polypropylene patch.

Repair with patch is the most widely used method in the treatment of hernia due to both reduction patient discomfort after surgery and the possibility of recurrence of the hernia [Rodgers et al., 2000, Arroya et al., 2001].

Synthetic materials can be used safely first in 1959 [Klosterhalfen et al., 2005] and developing of different materials have been released until today. There is no consensus ideas between researchers and clinicians about which material is ideal patch. Removal of the PTFE mesh from related region of the body in the case of infection inevitable. Incisional hernia recurrence is observed in 50% after primary repair. Incisional hernia repair with mesh in which a standard treatment is now recognized by all surgeons [Leber et al., 1998].

Composite meshes widely used in the treatment of hernia is the most referenced treatment methods. When the difficulties encountered in the use of existing composite meshes consider, the main problems faced in almost all outdoor operations are occurrence of infections, bleeding, biomaterials and tissue incompatibility. In this area, different structures and features developed by polymers and biomaterials market defines ‘composite patch’ which has given promising results in tissue-biomaterial compatibility, however, ideal mesh with the desired characteristics have not been developed yet. Composite meshes are used in both intraperitoneal and extraperitonal operations. The composite meshes used in intraperitoneal operation are usually bilayer and it is originated a non-biodegradable layer such as PP and the other layer consists of minimizing visceral adhesion risk. Because visceral adhesion occurs in the first 1-2 weeks of the postoperative period, the other side may comprise biodegradable polymers as well as permanent ones. The composite meshes used in extraperitoneal operations have generally a homogeneous composite structure to indicate the same properties throughout the material on abdominal wall.

Electrospun method is an appropriate method to obtain high surface/volume ratio [Lee et al., 2006]. In this method, nanofibers with desired thickness and pore size can be practically obtained by changing the voltage, solution concentration, flow rate, the distance between needle tip and collector. In addition, porosity is an important parameter for tissue growth and regeneration therefore, it makes this method advantageous.

In the U.S. Pat. No. 5,002,551, oxidized regenerated cellulose fabric is used for prevention of intraperitoneal adhesions after surgery and then, to determine the impact, rabbits were sacrificed at the end of the 2 weeks of the in vivo study and it was reported that adhesion was decreased. In the EU. Patent No. 0 262 890 A2, oxidized regenerated cellulose containing heparin is designed to be used as adhesion preventive membrane. This membrane provided a physical barrier properties between area of surgical activity and neighboring tissue, also indicated that it is not toxic and has anti-adhesion effect.

In the U.S. Pat. No. 5,364,622, hydroxyethyl cellulose hydrogel which has comprising tissue plasminogen activator (tPA) is used to prevent adhesion of organs which may occur after the operation and it stated to possess antiadhesive effect.

The U.S. Pat. No. 6,630,167 B2 describes foam of foam solution containing non crosslinked hyaluronic acid, crosslinked hyaluronic acid and aqueous solution mixture of both. Antiadhesive membranes was obtained from these solutions.

In the U.S. Pat. No. 6,150,581 describes a method in which defect area in dog abdominal wall is coated with the alginate solution by spraying method and after that the surface region coated with alginate is again coated with chitosan solution to get rid of adhesion in application area.

In the U.S. Pat. No. 5,795,584, films which generated from synthesized biodegradable copolymer is placed on polypropylene (PP) mesh by impression with PTFE coated plate and thus it is obtained as a multilayer barrier. The polymers used in this study are glycolide/trimethylene carbonate, glycolide/lactide, glycolide/lactide/trimethylene carbonate copolymers.

The U.S. Patent No. 2001/0008930 A1 and U.S. Pat. No. 5,614,587 studies, collagen which is a protein having biocompatible, non-toxic and biodegradable feature is used as post operative adhesion prevention material.

In the WO Patent No. 2007/029913 A1, multilayer membranes which has hydrophilic and hydrophobic layers containing nano size fibers and antiadhesive property is designed by electrospun method. The hydrophobic layer consist of at least one of peptide, amino acid, polysaccharide, polyorthoester, polycarbonate, polyamide esters, polyalpha-cyanoacrylate and polyphosphazene.

The U.S. Patent No. 2002/0173213 A1 discloses biodegradable and/or absorbable materials obtained as fiber from polymer via electrospun method in order to reduce post-operative adhesion. This study uses glycolide, lactide, dioxane, caprolactone, trimethylene carbonate and comprises ethylene glycol and lysine as bioabsorbable monomers and also these monomers can be used to be homopolymer or copolymer.

The CN Patent No. 102908677A describe a hernia patch containing polycaprolactone (PCL) on a polylactic-co-glycolic acid (PLGA) layer obtained by electrospun method to diminish adhesion in applied area.

WO 2008/075398 A2 involves coating by electrospinning method on polypropylene mesh. This mesh is coated on one or two surfaces with a film of polymer material that will prevent the formation of tackiness and/or reduce the occurrence of erosion. According to another embodiment, the mesh is coated with a polymer fiber web. The coating network of polymer fibers and nano fibers was realized by electrospinning technique. Although many polyesters and polysaccharides have been proposed for this method, the neutral solution has not been mentioned in an effort to neutralize chitosan, which is an important parameter, especially by antibacterial effect, and has not been suggested in a non-acidic solvent.

Hydrogels and solid films used as adhesion barriers have been introduced on the market for more than 30 years. On the other hand, combination of a standart mesh and material with anti adhesion property is a much more new area. Biodegradable or non-biodegradable meshes which can be single or multiple layer are used to prevent adhesion. However, these materials have not antibacterial and/or hemostatic properties to prevent post-operative complication.

### SUMMARY

In order to solve the aforementioned problems, this study is aimed to prevent post-operative complications such as infection, adhesion formation and bleeding by means of newly develop multifunctional biomaterials which has anti adhesive, antibacterial and hemostatic properties.

Developed products can be applied in the industrial field besides their antibacterial, hemostasis and anti adhesive properties. Thus, resulting products which have these properties are add significance to the invention.

The best mode of the invention is designed composite layer which is PLGA blend system containing by weight of 10% and 30% chitosan on PP mesh. However, the mentioned examples should not be limited.

The anti-adhesion barrier feature of present invention is useful regarding industrial applicability. The multi layered meshes which has non-toxic, antibacterial, hemostatic and tissue regeneration properties can solve the problems of post operative complications and thus gives the best convenience results when applied to operation areas.

Two types of composite mesh which can be used for intraperitoneal and extraperitoneal hernia surgery were developed. Both mesh accelerate tissue regeneration and have anti-adhesive, antibacterial, hemostatic properties. Products are describe below; For intraperitoneal applications; bilayer membranes were obtained with coated polymeric blend systems prepared with polyester-based polymers and chitosan by electrospun method on the PP mesh woven material that has specific pore spacing. Polyester-based polymers can be polyglycolic acid, polylactic acid, polyglycolic-co-lactic acid, polytrimethylenecarbonate, polyglycolic-co-trimethylene carbonate, polylactic-co-trimethylene carbonate, polycaprolactone, polyglycolic-co-caprolactone, polylactic-co-caprolactone, politrimetilencarbonate-co-caprolactone.

For extraperitoneal applications; composites meshes were acquired by weaving of mono or multi-filaments polyester, polypropylene based yarns and coated with chitosan.

### BRIEF DESCRIPTION OF THE DRAWINGS

FIG. 1. Microscope image of PP mesh obtained from the monofilament yarn

FIG. 2. Microscope image of composites meshes acquired by weave of multi-filaments polyester, polypropylene based yarns

FIG. 3. Cross section SEM images of monofilament PP mesh

FIG. 4. Cross section SEM images of monofilament PP mesh coated with polysaccharides by electrospun method

FIG. 5. Surface SEM images of PP mesh coated with polysaccharides by electrospun method

FIG. 6. Surface SEM images of PP Mesh coated with PLGA/Chitosan % 50 by electrospun method

FIG. 7. Implant figures of PP mesh coated with polyester/polysaccharide blend system by electrospun method

FIG. 8. Photographs of in vivo studies containing PP mesh implant

FIG. 9. Photographs of in vivo studies of PP mesh coated with PLGA/Chitosan % 30

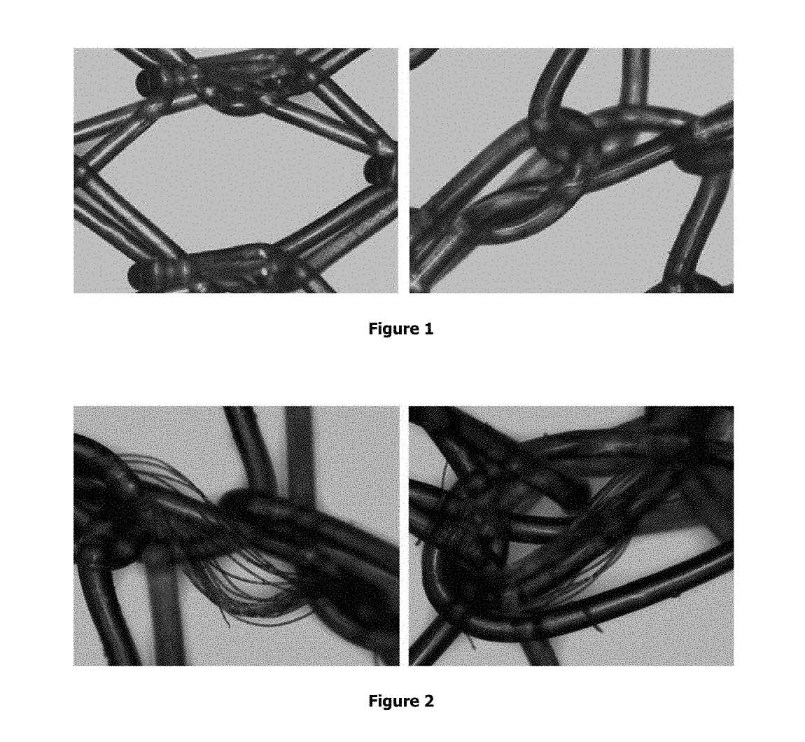

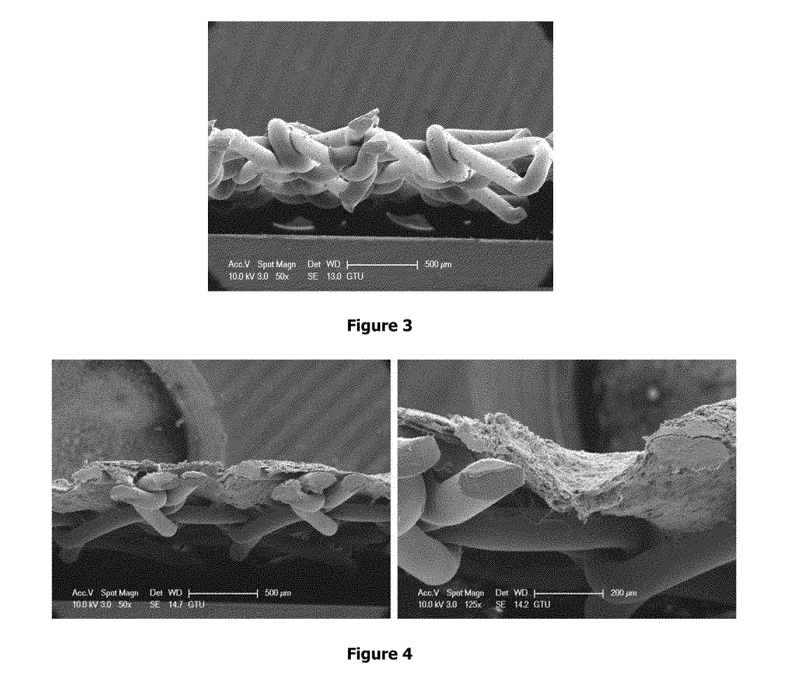

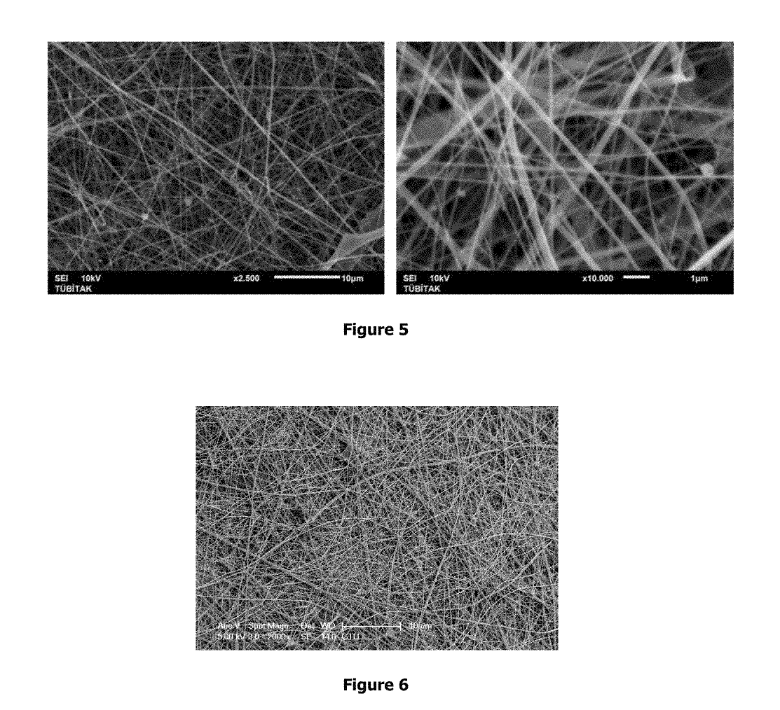

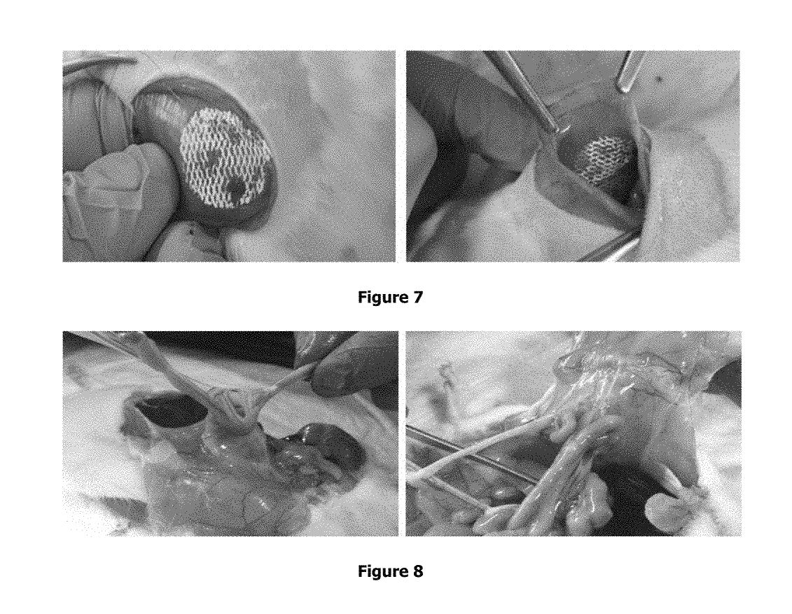

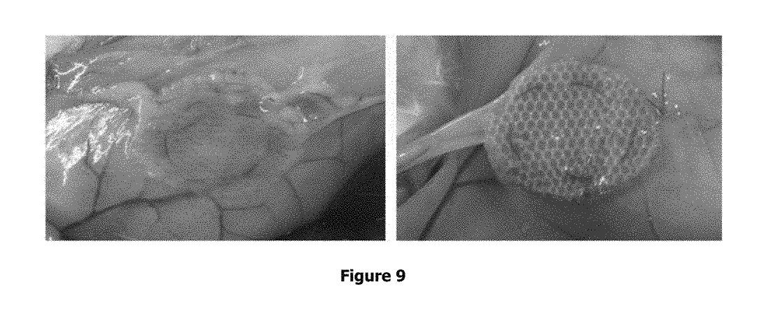

### DETAILED DESCRIPTION OF THE EMBODIMENTS

In the present invention, biocompatible, antibacterial, hemostatic and anti-adhesive meshes for intraperitoneal and extraperitoneal applications were developed. Details of production steps and resulting products are indicated below.

Process Steps:

Step 1. Preparation of Solutions and Blend System
4% weight solution of chitosan and PLGA was prepared in HFIP solvent and stirred with a magnetic stirrer for 12 h at ambient conditions. Polysaccharide ratio is about 30% of the total material in prepared blend system. The filtration was performed to remove the dissolved chitosan particles.
6% weight solution of chitosan and PLGA was prepared in HFIP solvent, stirring with a magnetic stirrer for 12 h at ambient conditions. Polysaccharide ratio is about 10% of the total material in prepared blend system. The filtration was performed to remove the dissolved chitosan particles.

Step 2. Application of Electrospun Blend System
Solution and blend systems were put into syringe and it was placed in a pump of electrospun device. PP mesh was coated on the aluminum cylinder then; optimized parameter values of voltage, the distance between needle tip and collector and flow rate were applied. Fiber structure was homogeneously collected on the PP mesh with the rotation of the aluminum cylinder. After the desired amount of product collected, bilayer mesh is completely dried at 40° C. vacuum oven for 1 day.
The present invention is detailed in the above examples, but is not limited to the examples described herein.

1. Bilayer intraperitoneal mesh, having biocompatible, antibacterial, hemostatic and anti-adhesive properties and certain thickness and porosity was prepared with combination of PP Mesh and blend system of biodegradable polyester-based polymers and polysaccharides coated on PP layer as nanofiber via electrospun technique:
a) Polyester/polysaccharide nanofiber structure and,
b) Scaffold system derived from mono or multiflament polypropylene yarn.

2. Double and/or triple-layer extraperitonal composite mesh, which has biocompatible, antibacterial, hemostatic, and antiadhesive properties and certain thickness and porosity, was prepared with blend systems of biodegradable polyester-based polymers and polysaccharides coated on PP/polyester woven material:
a) Biodegradable mono or multi-filament polyester/polypropylene yarn and;
b) Scaffold coated with polysaccharides.

3. According to claims 1 and 2, wherein polyester is polyglycolic acid, polylactic acid, polyglycolic-co-lactic acid, polytrimethylenecarbonate, polyglycolic-co-trimethylene carbonate, polylactic-co-trimethylene carbonate, polycaprolactone, polyglycolic-co-caprolactone, polylactic-co-caprolactone, politrimetilencarbonate-co-caprolactone.

4. According to claims 1 and 2, wherein polysaccharide is chitosan, chitin, starch, alginate, hyaluronate, and glycogen.

5. According to claims 1 and 2, wherein polyester/polysaccharide part is between 1-99% by weight and polyester part of the blend system has thickness of 1 to 999 micron while total thickness of mesh is from 1 to 1000 microns.

6. According to claims 1 and 2, wherein multi or mono polypropylene yarn diameter is from 1 to 500 microns and the number of multi filament polypropylene yarns is from 2 to 100.

7. According to claim 1, wherein nanofiber diameter of polyester/polysaccharides layer coated on PP mesh is from 10 to 1000 nm.

8. According to claim 2, wherein coating methods for PP/polyester woven material are elektrospun, spray, dip coating and cast method.

9. According to claim 2, wherein polyester filament number for PP/polyester woven material is from 1 to 100.

10. According to claim 8, wherein the device voltage is from 1 to 40 kV, the distance between the needle tip and collector is from 2 to 40 cm, flow rate is from 2 to 5000 microliters/minute.

11. According to claim 8, wherein porosity of polyester/polysaccharide layer is between 10-80% and pore size of polyester/polysaccharide layer is from 10 nm to 10 microns.

12. According to claim 8, wherein solvent used for biodegradable polyester/polysaccharide blend system is a mixture of volatile, polar and non-polar organic solvent and acetic acid or organic solvent and trifluoroacetic acid (TFA).

13. Organic solvents used in this study are indicated in the following list but these solvents are not limited to this list. According to claims 11 and 8, wherein organic solvent is hexafluoro isopropanol (HFIP), dichloromethane, chloroform, dimethylformamide (DMF), tetrahydrofuran (THF), dioxane, dimethylsulfoxide (DMSO), acetone, acetonitrile, 1-butanol, 2-butanol, 2-butanone, t-butyl alcohol, carbon tetrachloride, chlorobenzene, cyclohexane, 1,2-dichloro ethane, diethylene glycol diethyl ether, diethylene glycol diethyl ether, 1,2-dimethoxyethane, ethanol, ethylacetate, ethylene glycol, glycerine, heptane, hexamethylene phosphoramide, hexane, methanol, methyl t-butylether, methylene chloride, N-methyl-2-pyrrolidinone, nitromethane, pentane, petroleum ether, 1-propanol, 2-propanol, pyridine, toluene, triethylamine, water, o-xylene, m-xylene, p-xylene.

14. According to claim 11, wherein volume of acetic acid or TFA in organic solvent/acetic acid, or organic solvent/trifluoroacetic acid mixture is between 1-90%.

15. According to claim 13, wherein solvent used that can be at least one or a mixture of more than one in polyester, polysaccharide or polyester/polysaccharide solution.

16. According to claim 8, wherein polyester/polysaccharide solvent used, weight ratio of polysaccharide to polyester, is between 0.1 to 99.1% and the molecular weight of polyester (Mₙ) is from 10.000 to 1,000,000 Da.
